(19) United States
(12) Patent Application Publication
(10) Pub. No.: US 2009/0017528 A1
(43) Pub. Date: Jan. 15, 2009

(54) Nucleic Acid Hybridization Device

(76) Inventors: Pei-Tai Chen, Keelung City (TW); Ji-Yen Cheng, Taipei (TW)

Correspondence Address: WPAT, PC;INTELLECTUAL PROPERTY ATTORNEYS, 2030 MAIN STREET, SUITE 1300, IRVINE, CA, 92614, US

(21) Appl. No.: 11/777,265

(22) Filed: Jul. 12, 2007

(51) Int. Cl. C12M 1/34

(52) U.S. Cl. 435/287.2

(57) ABSTRACT

The present invention relates to a hybridization device coupled by means of vibration to shorten the time needed for hybridization. This hybridization device of the present invention comprises an oscillation generating unit; a spacer attached to the top of the oscillation generating unit, which wraps around to form a tank, and the tank is loaded with a transmission solution; a loading plate having a loading face was paved with at least one nucleic acid probe, and the flip side of the loading face is a transmission face, which covers on top of the spacer to form a closed space, and is attached with the transmission solution. The vibration generated by the oscillation generating unit can be propagated to the loading plate through the transmission solution to promote the effective mixing of the nucleic acid to be analyzed and the probe, further to shorten the time needed for nucleic acid hybridization and to increase the detection sensitivity.

### BACKGROUND OF THE INVENTION

1. Field of the Invention

The present invention relates to a nucleic acid hybridization device, and in particular to a hybridization device coupled with oscillation generating units to shorten the time needed for hybridization and to increase the detection sensitivity.

2. The Prior Arts

Nucleic acid hybridization with a probe is one of the most popular methods used to identify the target DNA from a sample containing the desired gene or the nucleic acid fragment. Conventionally, the hybridization reaction was carried out by blotting or transferring the sample nucleic acid to a substrate such as a membrane, hybridizing and pairing the nucleic acid with a probe having specificity. The results of the hybridization are shown by the probe-marked present molecule in methods such as fluorescence, coloring method, chemiluminescence method, and radiography method and so on.

The major conventional nucleic acid hybridization methods are Southern blotting for DNA analysis and Northern blotting for RNA analysis. The nucleic acid samples to be analyzed were transferred to the membrane after electrophoresis separation then hybridized with the nucleic acid probes. Therefore, the conventional hybridization reaction has to go through procedures such as electrophoresis separation, transferring steps and the hybridization of probes, which needs more than 10 hours for the reaction time. In addition, other blotting method in which the nucleic acid to be analyzed is directly dropped is referred to as dot-/ slot-/ spot blotting method according to the purpose of the detection. The dot-/ slot-/ spot blotting method is usually applied among the abovementioned methods in general qualitative analysis or in large batches of analysis, because the analyzing time can be shortened due to the needlessness of electrophoresis for separation and transfer, and the cost is low due to the needlessness of electrophoresis and transferring devises and various related agents.

The abovementioned methods are the most widely used conventional methods for nucleic acid detection. However, manifold and numerous devices are needed for electrophoresis, transblotting, nucleic acid fixation, temperature controlling and shaking in these methods, which could only be carried out in a laboratory. On the other hand, various solutions and reagents are needed for each step, which is therefore costly. Hence, the cost and time for the conventional analysis methods are non-economical.

To solve the problems of the abovementioned methods, some detection methods used microarray chips or microfluidic chips for analysis. Among them, the laboratory functions such as sample feeding, mixing, separation, heating and detection can be integrated into a microfluidic chip. Therefore a microfluidic chip system is also called as: Lab-on-a chip. The ability to perform laboratory operations on small scales in the chips makes the devices needed, the reagents, solution used be downsized at the same time. Therefore the costs for space, energy sources, the materials, solution and reagents can all be lowered remarkably. Most importantly, the time needed for the experiment, even the detection sensitivity can be improved greatly.

Refer to FIG. 1 for the conventional microarray chip applied in nucleic acid analysis. There was a mixing apparatus combining microarray chips and piezoelectric actuator to enhance the mixing efficiency of solution as the nucleic acid hybridization device 10. The vibration generated by the piezoelectric actuator 15 enhances the interactions of the solution to mix thorougly in the hybridization device 10. The nucleic acid hybridization device 10 comprises a vessel base 11, a nucleic acid probe 121 spot-printed gene microarray chip 12 which is set inside the vessel base 11, a spacer 13 on top of the gene microarray chip 12, a tank 131 which is formed and surround by the spacer 13, a piezoelectric actuator 15 containing fastening element 14 covered on top of the tank 131 to form a closed space. The reaction solution 132 containing the nucleic acid to be analyzed can be injected into tank 131 through the inlet/outlet hole 141 and contacts with piezoelectric actuator 15.

The piezoelectric actuator comprises special piezoelectric materials, which can generate the ultrasonic vibration via piezoelectric effect, and be transmitted through surface acoustic wave. Fluid can be driven to move using surface acoustic wave since it is propagated through Rayleigh wave, and Rayleigh wave is a traveling wave. Therefore, reaction solution 132 come into contact with piezoelectric actuator 15 will be shaken and flown when piezoelectric actuator 15 is vibrating. The flow field generated is shown in FIG. 2A and 2B. The speed for fluid along the surface of gene microarray chip 12 will be very high, increase the mixing and contacting chances for fluid and the probe attached to the surface. Somehow the nucleic acid to be assayed in the fluid displaces in big distance along the surface, which decreases the hybridization effect for nucleic acid probe 121. On the other hand, the piezoelectric actuator 15 in this hybridization device 10 attaches the reaction solution 132 directly, which may cause contamination even after proper washing. The accuracy for detection will be affected. In addition, the gene microarray chip 12 has to be packed first in the construction of the device, and then other components are covered or fastened one after another. The steps for construction are not only inconvenient, time-consuming, but also are easy to contaminate the inside of the vessel 11. The accuracy for detection will be affected once again.

### SUMMARY OF THE INVENTION

In order to simplify the construction steps in the application of the nucleic acid hybridization device, lower the chances for contamination, and further increase the sensitivity of detection, the present invention herein provides a nucleic acid hybridization device using a piezoelectric actuator, in which contamination can be prevented and the construction steps are simplified. The drawbacks of the conventional device are improved, and the mixing effects of the reaction solution are increased to further increase the sensitivity with improvement of the configuration of the device.

To fulfill the above objectives, the present invention provides a nucleic acid hybridization device comprising:
(1) an oscillation generating unit;
(2) a spacer attached to the top of the oscillation generating unit, which wraps around to form a tank, and is loaded with a transmission solution;
(3) a loading plate, which can be paved with at least one nucleic acid probe, and the flip side of the loading face is a transmission face, which covers on top of the spacer to form a closed space, and is attached with the transmission solution;
(4) an attaching layer; and,
(5) a reaction body connected to the loading plate with the attaching layer, the nucleic acid probes are paved to surround the inside of the reaction body.

The oscillation generating unit includes, but is not limited to, an electrostatic actuator, an electro-thermo actuator, an electro magnetic actuator, a surface acoustic wave actuator, or a piezoelectric actuator. The surface of the oscillation generating unit can be coated with a hydrophobic layer to improve the effect of vibrating propagation. The hydrophobic layer can be, but is not limited to, a Teflon layer.

The loading plate in the present invention includes, but is not limited to, a microarray chip, or any other nucleic acid to be analyzed laid- or fastened-plates. The reaction body covered on top of the loading plate is to form a space on the loading face of the loading plate to load the solution containing nucleic acid to be analyzed for hybridization. The structure of the reaction body is not particularly restricted therefore. To increase the hybridization efficiency and washing simplicity, a microfluidic chip can be used in the reaction body, in which the microfluidic channel can be set on the top of the nucleic acid probe. Fewer reaction solution or reagents can be used in the following reaction, and the time can be shortened to increase the detection sensitivity.

The vibration generated by the piezoelectric actuator of the hybridization device according to the present invention can be propagated to the loading plate through the transmission solution and further to the reaction solution. The reaction solution will be mixed better due to the vibration. The reaction time can be shortened; the chances for base pairing are increased to further increase the detection sensitivity.

On the other hand, the piezoelectric actuator and the transmission solution in the hybridization device according to the present invention will not be able to contact the reaction solution. The nucleic acid probe loading plates can therefore be discarded after each test. It is not necessary to operate the washing step inside the device. The detection accuracy can be remarkably increased since the operation is simple and the probable contamination problem in the conventional device can be prevented.

The present invention is further explained in the following embodiment illustration and examples. Those examples below should not, however, be considered to limit the scope of the invention, it is contemplated that modifications will readily occur to those skilled in the art, which modifications will be within the spirit of the invention and the scope of the appended claims.

### BRIEF DESCRIPTION OF THE DRAWINGS

The related drawings in connection with the detailed description of the present invention to be made later are described briefly as follows, in which:

FIG. 1 is a schematic diagram of a prior art.

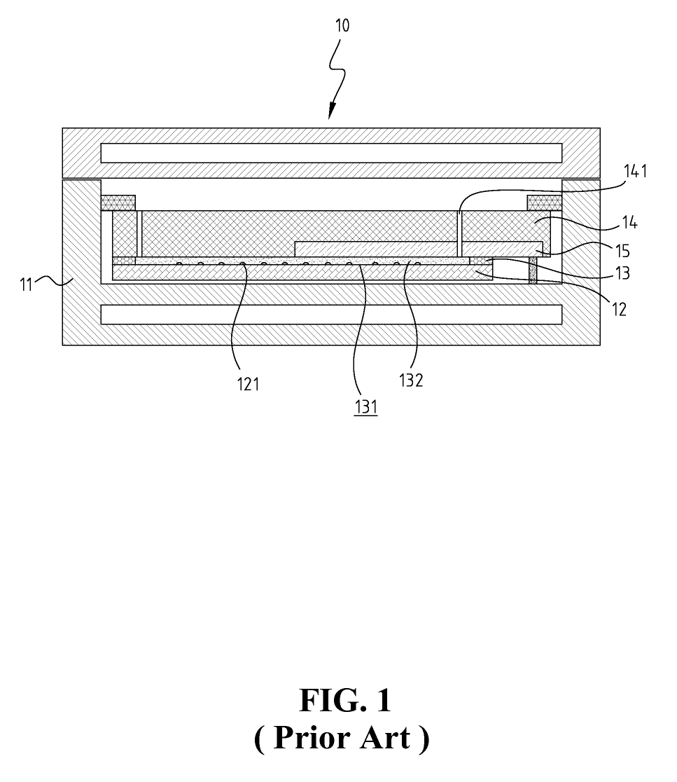

FIG. 2A and FIG. 2B are diagrams of flow field of the prior art.

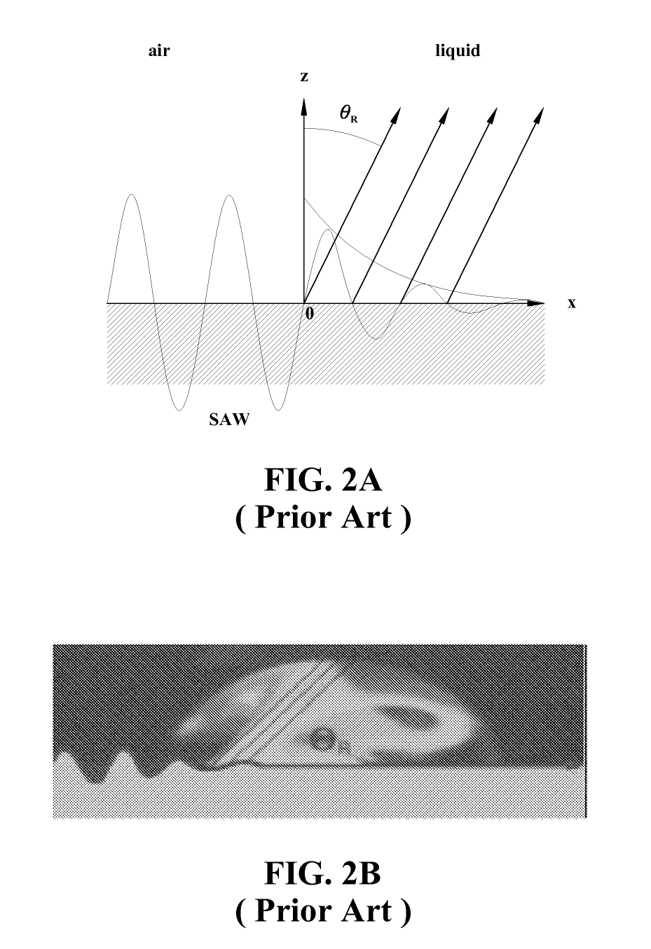

FIG. 3 shows the cross section diagram of the hybridization device in the embodiment according to the present invention.

FIG. 4 illustrates a perspective exploded view of the hybridization device in the embodiment according to the present invention.

FIG. 5A is a configuration diagram for the vibration transmission experiment of the hybridization device in the embodiment according to the present invention.

FIG. 5B illustrates the solution mixing results for the vibration transmission experiment of the hybridization device in the embodiment according to the present invention.

FIG. 6A shows the results before vibration generation for the nucleic acid hybridization experiment of the hybridization device in the embodiment according to the present invention.

FIG. 6B shows the results after vibration generation for the nucleic acid hybridization experiment of the hybridization device in the embodiment according to the present invention.

### DETAILED DESCRIPTION OF THE PREFERRED EMBODIMENT

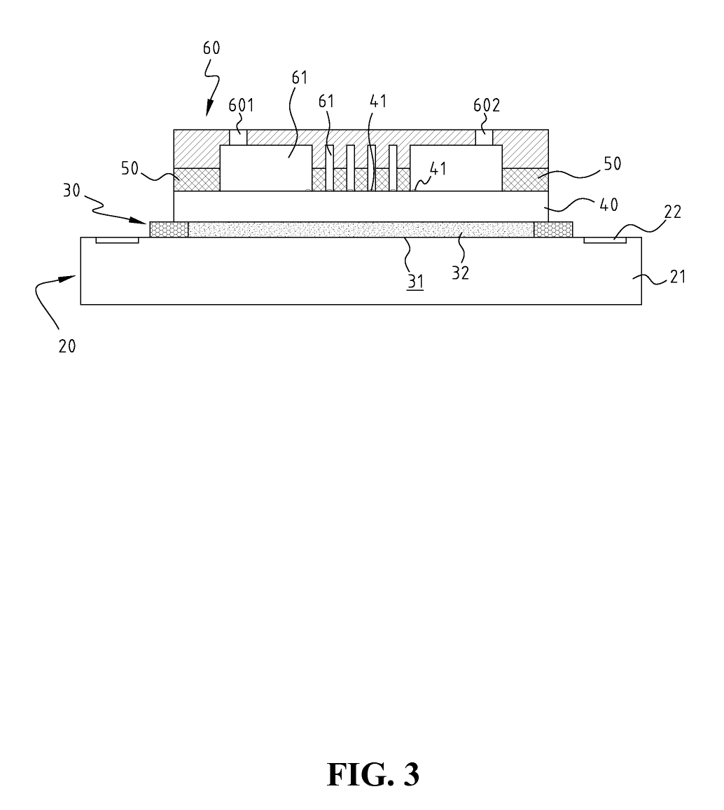

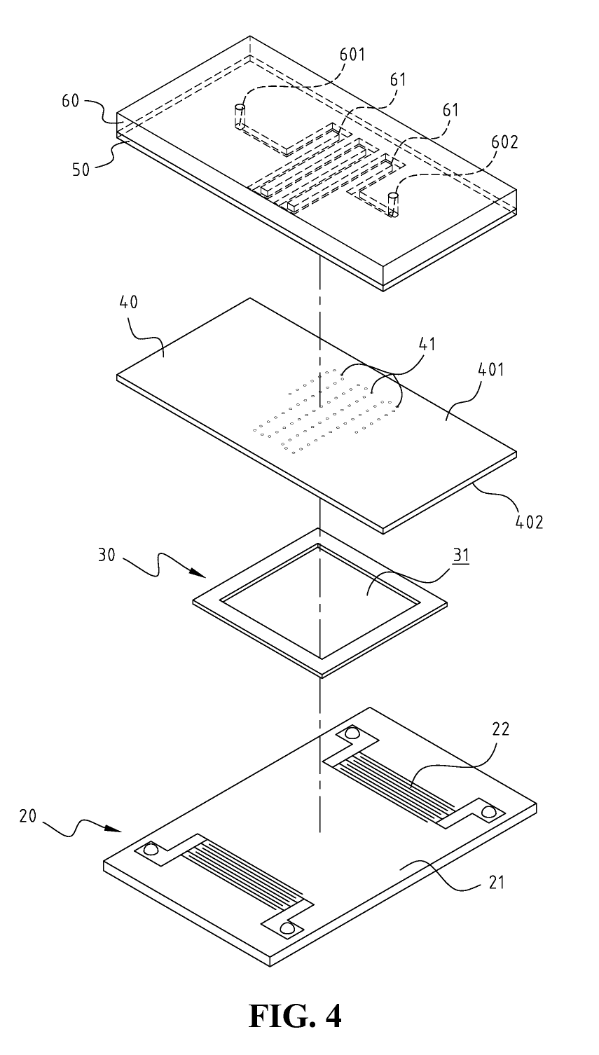

Referring to FIG. 3 and FIG. 4 at the same time, FIG. 3 shows the cross section diagram of the hybridization device in the embodiment according to the invention. FIG. 4 illustrates its perspective exploded view. The nucleic acid hybridization device of the present invention comprises an oscillation generating unit 20, a spacer 30, a loading plate 40, an attaching layer 50 and a reaction body 60, which are stacked together in an order. The vibration generated by the oscillation generating unit 20 can be propagated through transmission solution 30 to the loading plate 40 in order to promote the mixing effect and shorten the time needed for hybridization.

The purpose of using the oscillation generating unit 20 is to generate vibration, which is a piezoelectric actuator in the embodiment according to the present invention, can also be, but is not limited to, an electrostatic actuator, an electro-thermo actuator, an electro magnetic actuator, a surface acoustic wave actuator, or a piezoelectric actuator. The oscillation generating unit 20 can also include a piezoelectric substrate 21 and an interdigital transducer (IDT) 22, wherein the piezoelectric substrate can be a single crystal such as quartz, lithium niobate (LiNbO3), lithium tantalite (LiTaO3); a polycrystal such as lead zirconate titanate (PZT), barium-titanate (BaTiO3), zinc oxide (ZnO), and aluminum nitride (AlN); or, Polyvinylidene difluoride (PVDF) and the like, preferably lithium tantalite, which is not particularly restricted, but is preferred.

Electric field was applied to the piezoelectric substrate 21 through IDT 22 in the piezoelectric actuation method. The voltage difference existed in the surface of the piezoelectric substrate 21 induces strain through the piezoelectric effect, which deforms the piezoelectric substrate 21 and excites the acoustic wave along the surface of the piezoelectric substrate 21. The IDT 22 electrodes are manufactured with microelectromechanical method by using photomask defining the desired electrode pattern, followed by metal evaporation deposition or sputtering deposition after photolithography. In the embodiment of the present invention, acoustic wave can be generated between two IDT 22 in correspondence to the electrode polarity. The amplitudes of acoustic wave can be provided with different voltages according to needs, and can be in the range of 0.1 to 1 nm in general, but is not limited thereto.

The surface of the piezoelectric substrate 21 is hydrophilic, which causes viscosity drive to prevent the propagation of water and decrease transmission effect. Therefore the surface can be coated with a hydrophobic layer (not shown in the figure), which can be, but is not limited to, a Teflon layer. The hydrophobic coating can be carried out with a coating machine or other conventional methods.

The space 30 is set up between two IDTs to form the tank 31 for transmission solution 32 loading. The materials of spacer 30 for loading transmission solution 32 are particular restricted to a reliable seal and a good closing effect such as rubber, silicone, plastics and the like. The spacer 30 can be matched up to the amplitude of waves of the oscillation generating unit, which has a thickness of, but is not limited to, 0.1-1 mm, preferably 0.2 mm.

The loading plate 40 comprises a loading face 401 and a transmission face 402. The nucleic acid probe 41 can be paved or loaded on the loading face 401, while the transmission face 402 is connected with the spacer 30. The loading plate 40 is, but is not limited to, a gene microarray chip. The position of the nucleic acid probe paved or loaded on the loading faces is not particularly restricted, but is preferably on the top of the tank 31.

The attaching layer 50 is used to connect the reaction body 60 to the loading plate 40, which prevents leaking when reaction solution is loading into the reaction body 60. The attaching layer 50 is double-side adhesive, and the reaction body 60 is a microfluidic chip in the embodiment of the present invention, but both are not limited thereto. The attaching layer 50 can also be an adhesive agent or other media with closing effect; and the reaction body 60 can be a container to load the reaction solution. In the present embodiment, the twin adhesive used as an attaching layer 50 was connected to the microfluidic chip based on substrate of polymethyl methacrylate (PMMA). The PMMA was washed first before adhesion, and washed with ethanol in an ultrasound sonicator for 5 min, followed by deionized water washing to remove the remaining ethanol, and dried with nitrogen gas. The position of nucleic acid probe 41 was located and the pattern of microfluidic channel was designed and cut with a CO2 laser crafting system accordingly. The double-side adhesive on the surface of microfluidic channel was removed with laser cutting at a power of 3 W and a velocity of 48 mm/sec, followed by defocusing at a defocusing distance of 8 mm to continue the removal of double-side adhesive region (laser power 3.3 W, velocity 7 mm/sec). Further cutting was operated at a power of 22.5 W and a velocity of 31 mm/sec in the area of PMMA microfluidic channel.

The microfluidic chip was washed with ethanol for 5 min in an ultrasound sonicator after crafting. The debris was washed out with acetone and washed again with ethanol. After soaked in deionized water for 10 min, the microfluidic chip was dried with nitrogen gas and bake in an oven for 10 min at 70° C. to form the final product of microfluidic chip containing microfluidic channel 61, namely the reaction body 60.

The remaining double-side adhesive was removed from the microfluidic chip, and the microfluidic channel 61 was attached to the clean loading plate 40 (such as a slide) after treatment to finish the cohesiveness of double-side adhesive (attaching layer 50)/microfluidic chip (reaction body 60) and loading plate 40. The surface of microfluidic chip was set up a first inlet/outlet hole 601 and a second inlet/outlet hole 602, and can be connected with tubing and pumps to transport reaction solution.

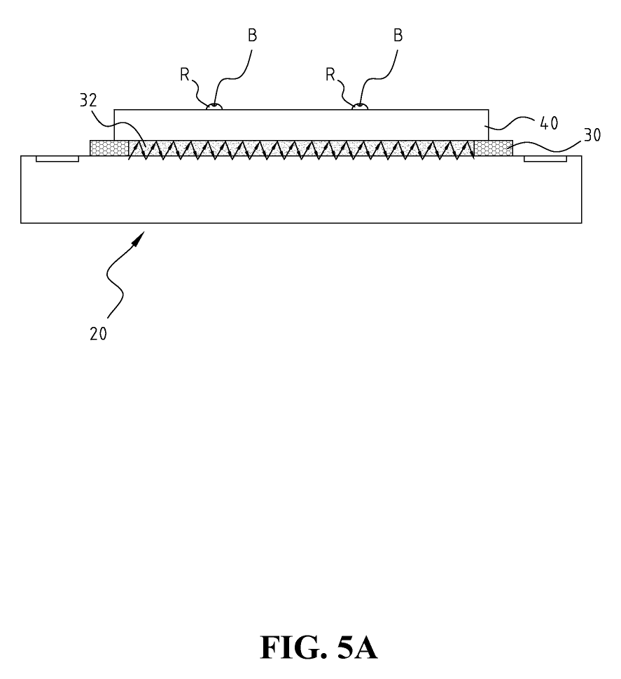

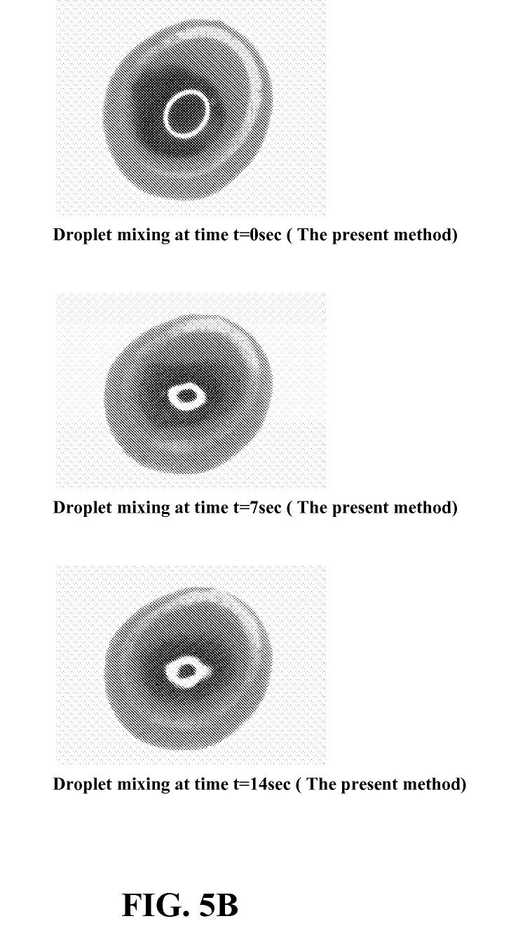

Referring to FIG. 5(A), it is a configuration diagram for the vibration transmission experiment of the hybridization device in the embodiment of the present invention. The surface pressure wave generated in the surface of the piezoelectric substrate 21 produced pressure waves at upward incline, which hit the transmission face 402 (the underside) of the loading plate and generated a reflection wave with the same angle of incidence. The purpose is to vibrate the loading plate 40 upward and downward, and to move the reaction solution filled inside the reaction body flowing around to promote the hybridization of nucleic acid probe 41 in the loading face 401. The oscillation generating unit 20, spacer 30 and the loading plate 40 are set up according to FIG. 3 in the embodiment, wherein the thickness of the spacer is 0.2 mm. The red dye R was dropped on the surface of loading plate 40, and a less amount of blue dye B was dropped onto the center of the red dye R before the experiment. The oscillation generating unit 20 was turned on to generate vibration at voltage of 50 volts to observe the result of mixing dyes, and search for the voltage needed for better vibration.

Referring to FIG. 5(B), the vibration transmission experiment of the hybridization device in the embodiment of the present invention makes the liquid inside the reaction body move upward and downward. An alternating current pulse signal of 50 volts was supplied to the finger electrode and the results of the droplet mixing at the 0, 7 and 14 second were shown. The droplet of blue dye stayed in the middle, not apparently mixed with the surrounding red dye, which confirms the upward and downward movement of liquid. Therefore the preliminary results of the nucleic acid device in the embodiment according to the present invention indeed can propagate the vibration generated by oscillation generating unit 20 to the surface of the loading plate 40, and shows great hybridization effect for solution in very short period.

### EXAMPLE 1

### The Time Needed for Nucleic Acid Hybridization Using the Hybridization Device of the Invention

To further explain the effect of the hybridization device of the present invention prepared according to the diagram of FIG. 3, the reaction body 60 using microfluidic chip was tested. The hybridization device comprised a microfluidic channel 61 with a width of 1 mm, a distance of 0.5 mm between channels, an area of 2.5 cm×5 cm, and a spacer with the thickness of 0.2 mm.

Firstly, 15 μM of the 80 mer nucleic acid probe was spot printed on the loading face 401 of the loading plate 40 by the conventional mechanical spotting with a robot. After spotting, the plate was allowed to connect with the reaction body 60 after the general steps of drying, crosslinking and pre-hybridization. The reaction body 60 (the microfluidic channel 61 containing microfluidic chip) produced as described above was adhered to the loading plate 40 with twin adhesive. The transmission solution 32 was then lead into the tank 31, and the connected reaction body 60 and the loading plate 40 were put on top of spacer 30 to be ready for hybridization reaction. The leading of transmission solution 32 was carried out by wetting the oscillation generating unit 20 with little transmission solution 32, and then the loading plate 40 was stacked on top. The transmission solution 32 exhibited surface tension effect to fill the tank. The transmission solution 32 is water in the embodiment according to the present invention, but is not limited thereto.

The nucleic acid to be analyzed needs to be labeled with a marker for detection after base paired with probe. The nucleic acid to be analyzed can be labeled with biotin in this embodiment, or using the radioactive isotopes ³²P or ³⁵S, for radioactive detection method, or using hexachlorofluorescein (HEX), Cy3 and Cy5 for fluorescence-based detection method, but is not limited thereto.

The biotin labeled nucleic acid to be analyzed, which can be base paired with probe 41 (the concentrations are 1 and 10 nM for control group and 0.5, 1 and 10 nM for experiment group respectively), was injected into the first inlet/outlet hole 601 in the reaction body 60 and reacted with probe 41 in the microfluidic channel 61. The DNA was maintained at 42° C. in a heating block for hybridization. The reactions were respectively performed for 20, 250, 480, and 1100 min without turning on the oscillation generating unit 20 (control group); or performed for 5, 10, 15, 20, 40 min with the oscillation generating unit 20 turning on (experiment group). The reaction body was washed with washing solution (2×SSC containing 0.1% (w/v) SDS) after hybridization, and subsequently fed with Cy5 labeled streptavidin for 5 min after washing to detect the fluorescence intensity under different conditions.

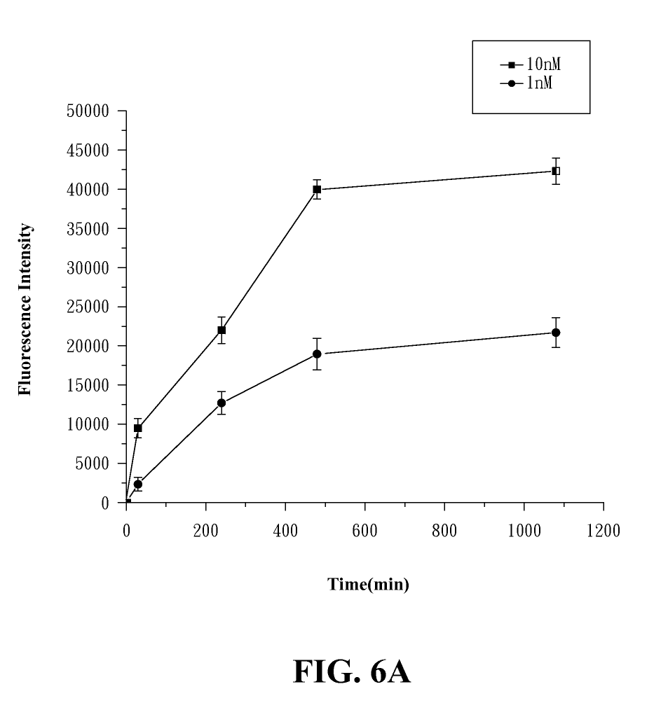

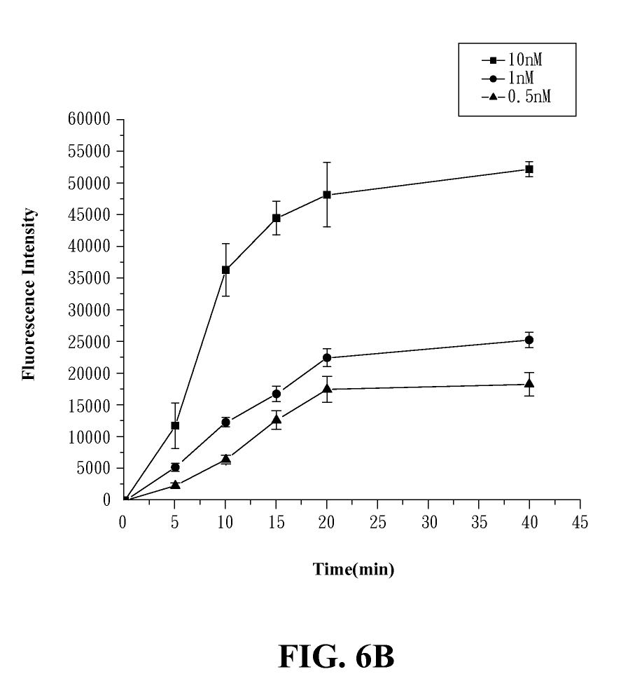

Referring to FIG. 6(A), results without vibration generation for the nucleic acid hybridization experiment of the hybridization device in the embodiment of the present invention. The fluorescence intensity reached saturation after 500 min of reaction with 1 nM or 10 nM of nucleic acid to be analyzed, that is, the hybridization becomes nearly saturated. And complete saturation was almost reached when reacted for 1100 min. The time needed for hybridization without vibration is pretty long.

In comparison, FIG. 6(B) shows results after vibration generation for the nucleic acid hybridization experiment of the hybridization device in the embodiment of the present invention. The fluorescence intensity curve nearly reached plateau condition after 20 min of reaction with 0.5 nM, 1 nM or 10 nM of nucleic acid to be analyzed, that is, the hybridization becomes nearly saturated. And the fluorescence intensity did not increase after 40 min of reaction. It is obvious that the nucleic acid hybridization device in the invention can shorten the hybridization time to around 25 folds, greatly decrease the time for nucleic acid detection. In addition, the nucleic acid hybridization device showed a higher fluorescence intensity after the vibration mixing effect is generated with the same concentration of nucleic acid. Therefore the detection sensitivity is also increased in the present invention.

What is claimed is:

1. A nucleic acid hybridization device, comprising:
(1) an oscillation generating unit;
(2) a spacer attached to the top of the oscillation generating unit and wraps around to form a tank, and the tank is loaded with a transmission solution;
(3) a loading plate having a loading face was paved with at least one nucleic acid probe, and the flip side of the loading face is a transmission face, which covers on the spacer to form a closed space, and is attached with the transmission solution;
(4) an attaching layer; and
(5) a reaction body connected to the loading plate with the attaching layer, the nucleic acid probes are paved to surround the inside of the reaction body, the reaction solution sits between the reaction body and the loading plate.

2. The nucleic acid hybridization device as claimed in claim 1, wherein the oscillation generating unit is an electrostatic actuator, an electro-thermo actuator, an electro magnetic actuator, a surface acoustic wave device, or a piezoelectric actuator.

3. The nucleic acid hybridization device as claimed in claim 1, wherein the surface of the oscillation generating unit is further coated with a hydrophobic layer.

4. The nucleic acid hybridization device as claimed in claim 3, wherein the hydrophobic layer is a Teflon layer.

5. The nucleic acid hybridization device as claimed in claim 1, wherein the transmission solution is water.

6. The nucleic acid hybridization device as claimed in claim 1, wherein the loading plate is a microarray chip.

7. The nucleic acid hybridization device as claimed in claim 1, wherein the attaching layer is a twin adhesive.

8. The nucleic acid hybridization device as claimed in claim 1, wherein the reaction body is a microfluidic chip.

9. The nucleic acid hybridization device as claimed in claim 1, wherein the reaction body is set up with at least one microfluidic channel, and the microfluidic channels are connected to the paved nucleic acid to be analyzed.

10. The nucleic acid hybridization device as claimed in claim 1, wherein the amplitude of waves of the oscillation generating unit is 0.1-1 nm.

11. The nucleic acid hybridization device as claimed in claim 1, wherein the spacer has a thickness of 0.1-1 mm.

12. The nucleic acid hybridization device as claimed in claim 10, wherein the spacer has a thickness of 0.1-1 mm.

13. The nucleic acid hybridization device as claimed in claim 11, wherein the spacer has a thickness of 0.2 mm.

14. The nucleic acid hybridization device as claimed in claim 1, wherein a closed space is formed between the reaction body and the loading plate, with the reaction solution inside.
